(19) United States
(12) Patent Application Publication
(10) Pub. No.: US 2021/0151186 A1
(43) Pub. Date: May 20, 2021

(54) ANIMAL HEALTH CHECKUP SUPPORT SYSTEM

(71) Applicant: FUJIFILM Corporation, Tokyo (JP)

(72) Inventors: Takeshi FUKUDA, Ashigarakami-gun (JP); Haruyasu NAKATSUGAWA, Ashigarakami-gun (JP)

(73) Assignee: FUJIFILM Corporation, Tokyo (JP)

(21) Appl. No.: 16/951,282

(22) Filed: Nov. 18, 2020

(30) Foreign Application Priority Data
Nov. 19, 2019 (JP) 2019-208889

(51) Int. Cl. G16H 50/20; G16H 40/20; G16H 10/60; G16H 10/40; G16H 50/70

(52) U.S. Cl. CPC G16H 50/20; G16H 40/20; A61B 6/508; G16H 10/40; G16H 50/70; G16H 10/60

(57) ABSTRACT

An animal health checkup support system comprises a breed information acquisition unit that acquires information pertaining to a breed of an animal as a target for examination and diagnosis, a blood examination result acquisition unit that acquires a result of a blood examination of the animal, a candidate disease specification unit that specifies candidate diseases as candidates of diseases, with which the animal is affected, using information pertaining to the breed and the result of the blood examination, and an additional examination suggestion unit that suggests an additional examination for use in diagnosis regarding whether or not the animal is affected with the candidate diseases specified by the candidate disease specification unit.

### CROSS-REFERENCE TO RELATED APPLICATIONS

This application claims priority under 35 U.S.C § 119(a) to Japanese Patent Application No. 2019-208889 filed on 19 Nov. 2019. The above application is hereby expressly incorporated by reference, in its entirety, into the present application.

### BACKGROUND OF THE INVENTION

### 1. Field of the Invention

The present invention relates to a system that supports a health checkup of a pet animal or the like.

### 2. Description of the Related Art

Hitherto, in a case of performing medical diagnosis, necessary clinical examinations are executed to obtain a determination material in diagnosis. As clinical examination (hereinafter, simply referred to as examination) that is executed at the time of diagnosis, a plurality of kinds of examinations, such as a blood examination, a urine examination, and an X-ray examination, are known, and one or a plurality of examinations are executed as necessary.

Furthermore, a system that integrally supports diagnosis in consideration of a plurality of examination results in a case where a human is treated as a target is known. For example, a system that acquires abnormal state names associated with a patient, counts examination names connected to clarified abnormal states and associated with abnormal states not clarified at present, and presents other necessary examinations using the count is known (JP2014-106776A).

### SUMMARY OF THE INVENTION

In a case where a target of diagnosis and examination is an animal (hereinafter, referred to as an animal), such as dog, cat, other pet animals (so-called pets), or domestic animals, other than a human, determination becomes complicated compared to a case where a human is a target of diagnosis and examination. This is because at least an animal species and a breed should be taken into consideration in a case where an animal is a target of diagnosis and examination.

Humans have a difference in race, such as Caucasoid, Mongoloid, or Negroid, and belong to single Homo sapiens as an animal species. Furthermore, there is no disease with which only a specific race is affected, no disease to which only a specific race is significantly predisposed, or the like, and humans substantially belong to a single breed. For this reason, in a case where a health state or the like of a human is diagnosed, there is no need to take a breed (race) into consideration, and examinations that should be executed are the same regardless of the breed (race).

On the other hand, animals have a difference in animal species, such as dog or cat, and susceptible diseases are different according to the animal species. For example, cat is predisposed to a disease pertaining to a kidney. Furthermore, even though animals belong to the same animal species, susceptible diseases are different depending on the breed. For example, among cats, a cat (for example, American Shorthair) of a specific breed is predisposed to a disease pertaining to a heart. That is, in a case where a target of diagnosis and examination is an animal, examinations necessary for appropriately diagnosing a health state or the like of the animal are different depending on the animal species and the breed. For this reason, there is a difficultly at the time of not only determination pertaining to a final health state or the like called diagnosis, but also determination regarding necessary examinations to obtain a determination material of the determination.

Accordingly, in an animal hospital that treats an animal as a target of diagnosis and examination, it is desirable to perform diagnosis support for suggesting necessary examinations to appropriately diagnose a health state or the like of the animal. Furthermore, in a case of supporting diagnosis using results of examinations relating to an animal, it is desirable to perform support with high likelihood taking the breed as well as the animal species into consideration.

An object of the invention is to provide an animal health checkup support system capable of performing support pertaining to diagnosis with high likelihood in a case of diagnosing a health state or the like of an animal.

An aspect of the invention provides an animal health checkup support system comprising a breed information acquisition unit that acquires information pertaining to a breed of an animal as a target of examination and diagnosis, a blood examination result acquisition unit that acquires a result of the blood examination of the animal, a candidate disease specification unit that specifies candidate diseases as candidates of diseases, with which the animal is affected, using information pertaining to the breed and the result of the blood examination, and an additional examination suggestion unit that suggests an additional examination for use in diagnosis regarding whether or not the animal is affected with the candidate diseases specified by the candidate disease specification unit.

It is preferable that the candidate disease specification unit specifies the candidate diseases within a range including at least a susceptible disease by specifying the susceptible disease of the animal using information pertaining to the breed and narrowing down the candidate diseases using the result of the blood examination.

It is preferable that the candidate disease specification unit specifies the candidate diseases within the range including the susceptible disease by selecting the candidate diseases using the result of the blood examination from within the range including the susceptible disease.

It is preferable that the candidate disease specification unit specifies the candidate diseases within the range including the susceptible disease by selecting the range including the susceptible disease from among the candidate diseases.

It is preferable that, in a case where the candidate disease specification unit specifies a plurality of the candidate diseases, the additional examination suggestion unit suggests an additional examination for each candidate disease.

It is preferable that the additional examination suggestion unit notifies of the candidate disease on which diagnosis regarding whether or not the animal is affected with the candidate disease is impossible and suggests an additional examination on the notified candidate disease.

It is preferable that the additional examination suggestion unit notifies of the candidate disease on which diagnosis becomes possible with an additional examination to be suggested and suggests the additional examination that makes diagnosis of the notified candidate disease possible.

It is preferable that the animal health checkup support system further comprises a storage unit that stores a past diagnosis result as a result of past diagnosis on the animal of the same or similar breed, and the candidate disease specification unit specifies the candidate diseases with reference to the past diagnosis result.

It is preferable that, in a case where there is the past diagnosis result on the animal of the same breed, the candidate disease specification unit specifies the candidate diseases with reference to the past diagnosis result on the animal of the same breed, and in a case where there is no past diagnosis result on the animal of the same breed, the candidate disease specification unit specifies the candidate diseases with reference to the past diagnosis result on the animal of the similar breed.

It is preferable that the animal health checkup support system further comprises an additional examination result acquisition unit that, in a case where an additional examination suggested by the additional examination suggestion unit is executed, acquires a result of the additional examination, the candidate disease specification unit specifies the candidate disease using the result of the additional examination in addition to information pertaining to the breed and the result of the blood examination, and the additional examination suggestion unit suggests an additional examination for use in diagnosis regarding whether or not the animal is affected with the candidate diseases specified using the result of the additional examination.

With the animal health checkup support system of the invention, it is possible to perform support pertaining to diagnosis with high likelihood or the like in a case of diagnosing a health state or the like of an animal.

### BRIEF DESCRIPTION OF THE DRAWINGS

FIG. 1 is an explanatory view showing a relationship between an animal hospital and an animal health checkup support system.

FIG. 2 is a block diagram showing an example of examination devices in the animal hospital.

FIG. 3 is a block diagram showing the configuration of an animal health checkup support system.

FIG. 4 is a table showing an example of animal species and breeds.

FIG. 5 is a table showing parts to be examined and execution frequencies of various examinations.

FIG. 6 is a table showing parts to be examined and execution frequencies of various examinations.

FIG. 7 is an explanatory view showing susceptible diseases.

FIG. 8 is a table showing an example of susceptible diseases according to a breed.

FIG. 9 is a flowchart showing the operation of the animal health checkup support system.

FIG. 10 is an explanatory view showing an aspect of support that is provided by the animal health checkup support system.

FIG. 11 is an explanatory view showing an aspect of diagnosis support of the modification example.

FIG. 12 is an explanatory view showing an aspect of diagnosis support of the modification example.

FIG. 13 is an explanatory view showing an aspect of diagnosis support of the modification example.

FIG. 14 is a flowchart showing the operation of an animal health checkup support system of a modification example.

FIG. 15 is a block diagram of an animal health checkup support system of a second embodiment.

FIG. 16 is a block diagram of an animal health checkup support system of a third embodiment.

FIG. 17 is a block diagram of an animal health checkup support system of a fourth embodiment.

FIG. 18 is an explanatory view showing a learned model that functions as a candidate disease specification unit.

FIG. 19 is an explanatory view showing a learned model that functions as a candidate disease specification unit and an additional examination suggestion unit.

### DESCRIPTION OF THE PREFERRED EMBODIMENTS

### First Embodiment

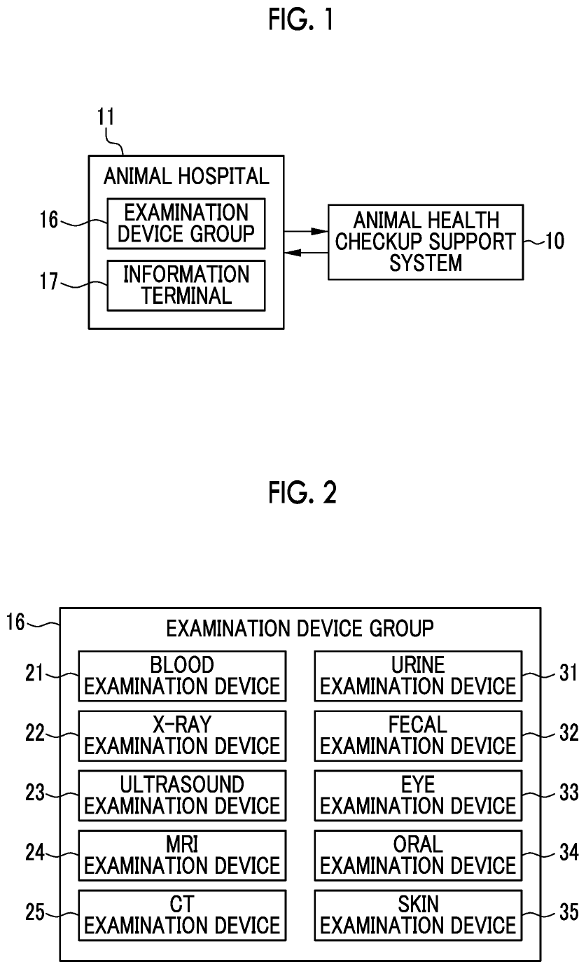

As shown in FIG. 1, an animal health checkup support system 10 is a system that cooperates with equipment of an animal hospital 11 to perform support (hereinafter, referred to as diagnosis support) for diagnosis and examination pertaining to health of an animal as an affected animal to a veterinarian of the animal hospital 11. An animal is an affected animal in the animal hospital 11, refers to general animals excluding humans, and is, for example, a pet animal, such as dog or cat, or a domestic animal, such as cow or pig.

Diagnosis and examination (health checkup) pertaining to health include not only diagnosis or examination that confirms a health state of an animal but also diagnosis or examination pertaining to determination regarding whether or not an animal is affected with a specific disease and diagnosis and examination pertaining to determination regarding a progression degree of a specific disease. Furthermore, diagnosis support refers to providing information that can be used in determination at the time of diagnosis or examination. Information that can be used in determination at the time of diagnosis includes not only information that becomes a determination material in diagnosis (for example, information for reporting that the animal is likely to be affected with a specific disease) but also diagnosis information on behalf of a veterinarian (for example, information for declaratively reporting that an animal is affected with a specific disease). Furthermore, information that can be used in determination at the time of examination is, for example, a suggestion to the effect that an unexecuted examination necessary in diagnosis regarding whether or not an animal is affected with a specific disease should be additionally executed (hereinafter, referred to as a suggestion for an additional examination).

The animal hospital 11 has, as equipment, for example, an examination device group 16 that has one or a plurality of examination devices, and an information terminal 17 that manages medical charts and the like. The animal health checkup support system 10 acquires information necessary for diagnosis support by performing communication with at least one of the examination device group 16 or the information terminal 17 and performs diagnosis support using the acquired information.

As shown in FIG. 2, the examination device group 16 has, for example, at least one of a blood examination device 21, an X-ray examination device 22, an ultrasound examination device 23, a magnetic resonance imaging (MRI) examination device 24, a computed tomography (CT) examination device 25, a urine examination device 31, a fecal examination device 32, an eye examination device 33, an oral examination device 34, a skin examination device 35, or the like. The veterinarian decides the types of necessary examinations, items, and the like in consideration of a symptom and the like and executes examination using various examination devices described above. In particular, there is a case where a specific examination (for example, blood examination) that can be used to distinguish many diseases is executed without depending on the symptom and the like.

The animal health checkup support system 10 can acquire examination results output from various examination devices described above from various examination devices directly or through the information terminal 17. The types and the number of specific examination devices configuring the examination device group 16 may be different depending on the scale or the like of the animal hospital 11. Note that, for cooperation with the animal health checkup support system 10, the examination device group 16 includes at least a device pertaining to an examination that is used in diagnosis support by the animal health checkup support system 10, and one or more other examination devices. In the embodiment, an examination that is used in diagnosis support by the animal health checkup support system 10 is a blood examination. For this reason, the device pertaining to the examination that is used in diagnosis support by the animal health checkup support system 10 is the blood examination device 21, and the examination device group 16 has examination devices, such as the X-ray examination device 22, in addition to the blood examination device 21.

The information terminal 17 is a so-called computer, and comprises a display unit 101 (see FIG. 10 or the like), such as a liquid crystal display, and an operating unit (not shown) having a keyboard, a pointing device, and the like. The information terminal 17 is a device that manages medical charts and other kinds of information, and manages, for example, information regarding at least one of an identification number (ID), a name, a name of an owner, an animal species, a breed, an age, results of various examinations, findings, diagnosis results, or the like of an animal that becomes an affected animal. The information terminal 17 can acquire examination results of the examination devices automatically or manual inputs and displays necessary information on the display unit 101.

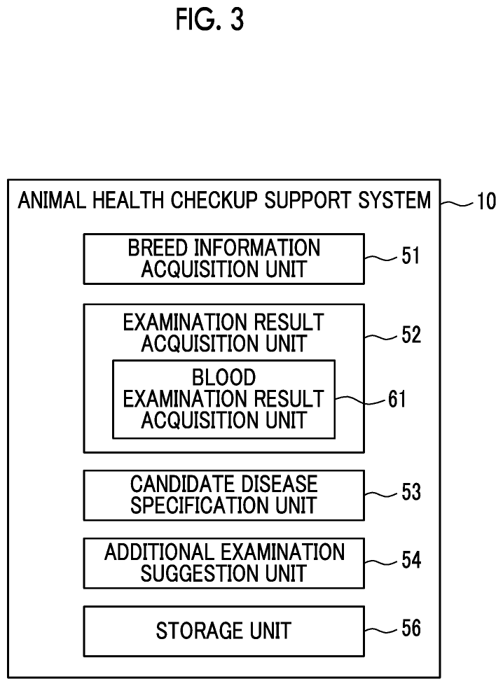

As shown in FIG. 3, the animal health checkup support system 10 comprises a breed information acquisition unit 51, an examination result acquisition unit 52, a candidate disease specification unit 53, an additional examination suggestion unit 54, a storage unit 56, and the like.

The breed information acquisition unit 51 acquires information pertaining to a breed of an animal as a target for examination and diagnosis. Information (hereinafter, referred to as breed information) pertaining to the breed information that can specify the breed directly or indirectly, such as a number or a symbol associated with the breed, in addition to a name representing the breed. In the embodiment, although the breed information acquisition unit 51 acquires the breed information through the information terminal 17, the breed information can be acquired from various examination devices of the examination device group 16, a management device of the examination device group 16, or the like, instead of the information terminal 17.

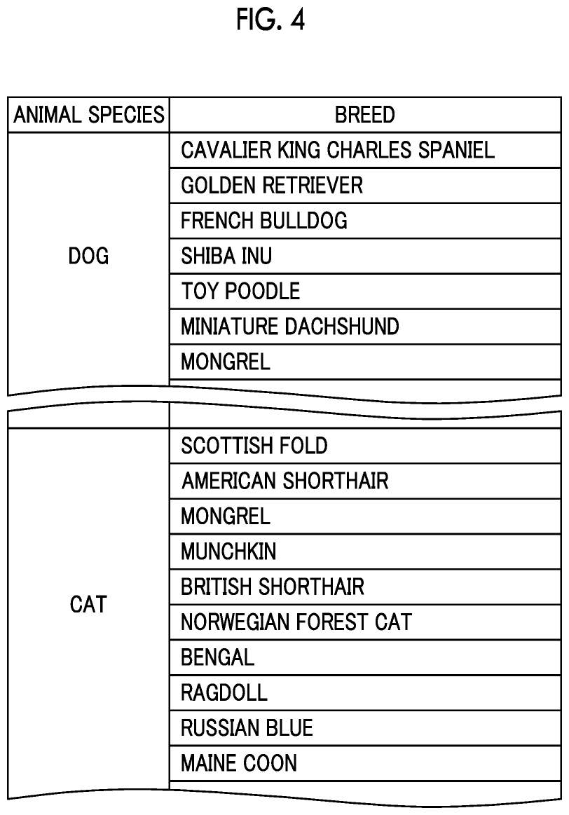

As shown in FIG. 4, the breed is a classification for subdividing a specific animal species in consideration of a genetic character or the like. For example, a classification for distinguishing between different species of intersterile animals, such as dog and cat, is an animal species. Then, for example, in regard to dog, Cavalier King Charles Spaniel, Golden Retriever, French Bulldog, and the like are breeds. Furthermore, the breeds include a “mongrel” made by mixing different breeds (mixed, hybrid, crossbred, or subspecies). Since the animal species and the breed have a relationship that the animal species is specified in a case where the breed is specified, the breed information includes information pertaining to the animal species, and also functions as information for specifying the animal species.

The examination result acquisition unit 52 acquires results of various examinations. The examination result acquisition unit 52 can optionally acquire a part or all of the examination results output from various examination devices of the examination device group 16. In a case where one examination device acquires examination results on a plurality of items, the examination result acquisition unit 52 can optionally acquire the examination results pertaining to a part or all of the items. Furthermore, the examination result acquisition unit 52 not only can acquire the examination results directly from various examination devices of the examination device group 16, but also can acquire the examination results through the information terminal 17 in a case where the examination results are registered in the information terminal 17.

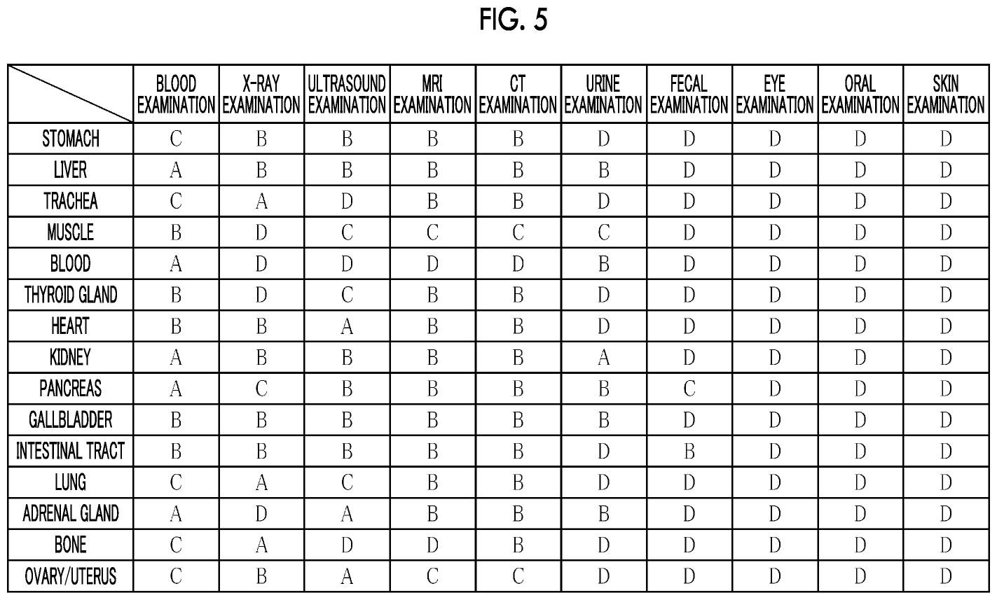

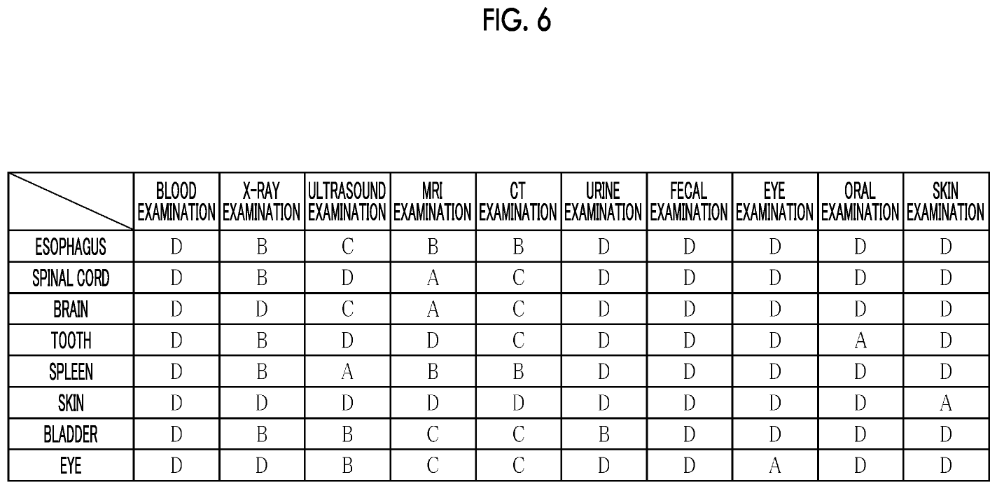

In the embodiment, the examination result acquisition unit 52 comprises a blood examination result acquisition unit 61 that acquires a result of a blood examination of the animal as a target for examination and diagnosis, and acquires at least the result of the blood examination to perform diagnosis support. While various examinations are optionally executed based on veterinarian's determination, as shown in FIGS. 5 and 6, the result of the blood examination becomes a determination material of the presence or absence and the degree of a disease pertaining to comparatively many parts (kidney, heart, and the like). The veterinarian substantially always executes the blood examination. Accordingly, the animal health checkup support system 10 of the embodiment acquires the result of the blood examination by the examination result acquisition unit 52 and performs diagnosis support based on the result of the blood examination. In FIGS. 5 and 6, evaluation “A” represents that an examination is particularly well executed for diagnosis of a disease pertaining to a part, evaluation “B” represents that the examination is well executed, evaluation “C” represents that the examination is sometimes executed, and evaluation “D” represents that the examination is not really executed.

The result of the blood examination includes values, evaluation based on comparison with reference values or reference ranges for determining a range of normality and abnormality, and the like of total protein (TP), albumin (Alb), globulin (Glob), an albumin-globulin ratio, aspartate aminotransferase (AST), alanine aminotransferase (ALT), alkaline phosphatase (ALP), gamma glutamyl transpeptidase (GGT), total cholesterol (TCho), triglyceride (TG), total bilirubin (TBil), glucose (Glu), amylase (Amy), lipase (Lip), urea nitrogen (BUN), creatinine (Cre), a BUN-Cre ratio, symmetric dimethylarginine (SDMA), phosphorus (P), Ca (calcium), sodium (Na), potassium (K), chloride (CI), and the like.

The candidate disease specification unit 53 specifies candidate diseases as candidates of diseases, with which the animal is affected, using the breed information and the examination results acquired by the examination result acquisition unit 52. In the examination result acquisition unit 52 of the embodiment, since the blood examination result acquisition unit 61 acquires the result of the blood examination, the candidate disease specification unit 53 specifies candidate diseases using the breed information and the result of the blood examination.

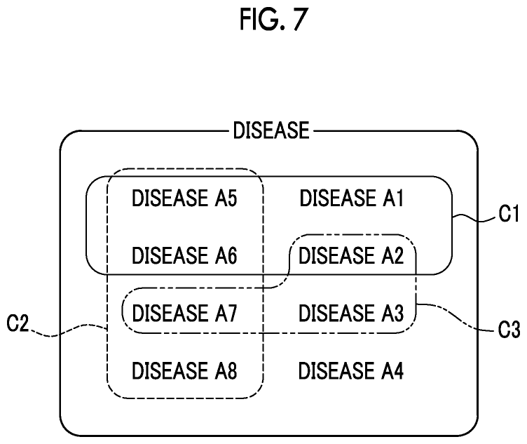

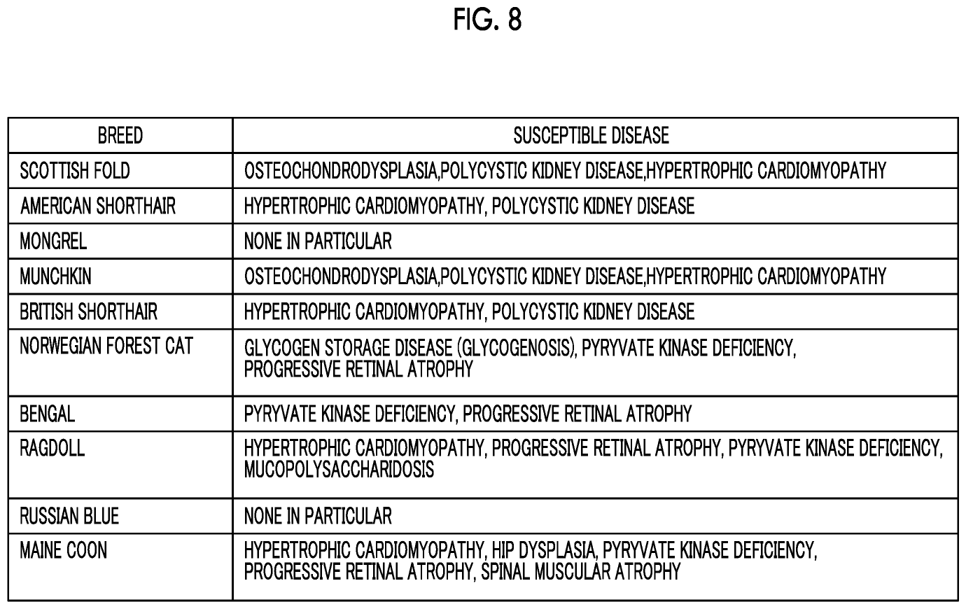

Furthermore, the candidate disease specification unit 53 specifies susceptible diseases of the animal using the breed information and narrows down the candidate diseases using the result of the blood examination, thereby specifying the candidate diseases within a range including the susceptible diseases. The susceptible diseases are diseases to which an animal of a specific breed is susceptible due to a genetic factor or the like. For example, as shown in FIG. 7, in a case where there are diseases A1 to A8 with which an animal is likely to be affected, an animal of a specific breed C1 is not predisposed to all diseases equally, and is susceptible to, for example, the disease A1, the disease A2, the disease A5, and the disease A6 due to a genetic factor or the like. Furthermore, an animal of a different breed C2 is susceptible to the disease A5, the disease A6, the disease A7, and the disease A8, and a breed C3 is susceptible to the disease A2, the disease A3, and the disease A7. In this way, in a case of an animal, the susceptible diseases are different depending on the breed. More specifically, for example, as shown in FIG. 8, in a case of cat, susceptible diseases of Scottish Fold are osteochondrodysplasia, polycystic kidney disease, and hypertrophic cardiomyopathy, and susceptible diseases of American Shorthair are hypertrophic cardiomyopathy and polycystic kidney disease. For this reason, regardless of the same animal species, a cat has different susceptible diseases depending on a breed. Accordingly, susceptible diseases are specified using the breed information and candidate diseases are specified within a range including one or a plurality of specified susceptible diseases, whereby it is possible to specify appropriate candidate diseases according to a breed of an animal. The range including the susceptible diseases refers to a set (group) of doubtful diseases including known susceptible diseases pertaining to a specific breed without omission, and may include diseases (for example, predisposed diseases determined depending on an animal species (quasi-susceptible diseases that are susceptible in proportion to susceptible diseases determined depending on a breed) and the like) other than known susceptible diseases determined depending on a breed. The range including the susceptible diseases can be determined in advance in one or a plurality of patterns according to data, such as a real affection rate, or empirically.

An animal of a specific breed may not have such susceptible diseases (for example, see “Russian Blue” of FIG. 8). Furthermore, since the degree of mixing of a plurality of breeds is not determined for a mongrel, there are no susceptible diseases determined depending on a breed called a mongrel. In a case where there are no susceptible diseases determined depending on a breed, the candidate disease specification unit 53 specifies all diseases, with which the animal as a target for examination and diagnosis can be affected, as susceptible diseases for convenience and narrows down candidate diseases. With this, it is possible to specify doubtful diseases from the result of the blood examination as candidate diseases without omission. Note that, in a case where there are no susceptible diseases determined depending on the breed, it is possible to specify susceptible diseases as the animal species determined from the breed instead of specifying all diseases, with which the animal can be affected, as susceptible diseases for convenience. For example, in a case of Russian Blue or a mongrel of a cat, while there are no susceptible diseases on the breed called the mongrel, an animal species called a cat is susceptible to diseases pertaining to a kidney than other animal species. For this reason, in regard to Russian Blue or the mongrel of the cat, the diseases pertaining to the kidney may be specified as susceptible diseases, and the candidate diseases may be narrowed down. In this case, it is possible to quickly specify the candidate diseases, and as a result, to perform quick diagnosis support (for example, real-time diagnosis support).

Furthermore, in the embodiment, the candidate disease specification unit 53 performs specification of the susceptible diseases based on the breed information and narrowing-down of the candidate diseases based on the result of the blood examination in this order. That is, the candidate disease specification unit 53 first specifies the susceptible diseases using the breed information, and then, selects diseases to be the candidate diseases from among the susceptible diseases using the result of the blood examination. With this, the candidate disease specification unit 53 specifies the candidate diseases within a range including the susceptible diseases. The susceptible diseases are determined depending on the breed, and the number of susceptible diseases is often smaller than the number of doubtful diseases from the result of the blood examination. In such a case, as described above, narrowing-down of the candidate diseases based on the result of the blood examination is performed after the specification of the range including the susceptible diseases based on the breed information, whereby it is possible to quickly specify the candidate diseases, and as a result, to perform quick diagnosis support.

The additional examination suggestion unit 54 decides whether or not an additional examination is necessary and which additional examination is necessary in performing diagnosis (hereinafter, referred to as the diagnosis of the candidate diseases) regarding whether or not the animal is affected with the candidate diseases specified by the candidate disease specification unit 53, and in a case where an additional examination is necessary, suggests the additional examination for use in the diagnosis of the candidate diseases. The suggestion of the additional examination performed by the additional examination suggestion unit 54 is diagnosis support in the embodiment. The additional examination is an examination for use in the diagnosis of the candidate diseases, and is an unexecuted examination or an examination including an unexecuted item. For this reason, the additional examination also includes an examination of which the result is already acquired by the examination result acquisition unit 52. For example, in a case where there is an unexecuted item among items necessary for the diagnosis of the candidate diseases, an examination of which the result is already acquired by the examination result acquisition unit 52 may be suggested as the additional examination.

In the embodiment, although the examination result acquisition unit 52 acquires the result of the blood examination by the blood examination result acquisition unit 61, for the diagnosis of the candidate diseases, in a case where an examination result is necessary on an item other than items included in the result of the blood examination already acquired, and a blood examination on the item is suggested as an additional examination. For example, in regard to hyperthyroidism of a cat, while the presence or absence of doubt can be determined with reference to the values of ALT and ALP, and the like included in the items of the blood examination, an examination result pertaining to serum total thyroxine (T4) as thyroxine is necessary for conclusive diagnosis. For this reason, in a case where hyperthyroidism becomes a candidate disease from the values of ALT and ALP, and the like, and the value of T4, and the like are not included in the result of the blood examination already acquired, the additional examination suggestion unit 54 suggests a blood examination on T4 as an additional examination. Furthermore, for example, in regard to hypothyroidism of a dog, while the presence or absence of doubt can be determined with reference to TCho included in the items of the blood examination, examination results pertaining to T4, free serum total thyroxine (FT4), and thyrotropin (c-TSH) are necessary for conclusive diagnosis. For this reason, in a case where hypothyroidism becomes a candidate disease from the values of TCho and the like, and the values of T4, FT4, and c-TSH, and the like are not included in the result of the blood examination already acquired, the additional examination suggestion unit 54 suggests a blood examination on the items as an additional examination. In addition, for both a cat and a dog, in regard to renal insufficiency, while the presence or absence of doubt can be determined based on the values of Alb, TCho, Glu, and BUN, and the like, additional examinations of total bile acids and ammonia (NH3) are necessary for conclusive diagnosis. Furthermore, in regards to diabetes and hypoglycemia, while the presence or absence of doubt can be determined based on the values of TCho and Glu, and the like, additional examinations of pancreas specific lipase, a trypsin-like immunoreactive material (TLI), insulin, and fructosamine are necessary for conclusive diagnosis.

Furthermore, the suggestion of the additional examination refers to providing information capable of making a veterinarian recognize that a specific additional examination should be executed, and for example, display of a message, alarming of speech, or the like to the information terminal 17 or the like can be performed. In the embodiment, the additional examination suggestion unit 54 suggests an additional examination by displaying a message on the information terminal 17.

The storage unit 56 is, for example, a database pertaining to diseases, and stores a plurality of diseases with which animals are likely to be affected, examinations and examination items necessary for diagnosis pertaining to each disease, a range of normality or abnormality of each examination item, and the like in association with one another. Furthermore, the storage unit 56 stores each disease in association with an animal species and a breed directly or indirectly. For this reason, the candidate disease specification unit 53 can specify the susceptible diseases pertaining to the breed using the breed information with reference to data of the storage unit 56. Furthermore, the candidate disease specification unit 53 can narrow down the candidate diseases using the result of the blood examination with reference to data of the storage unit 56.

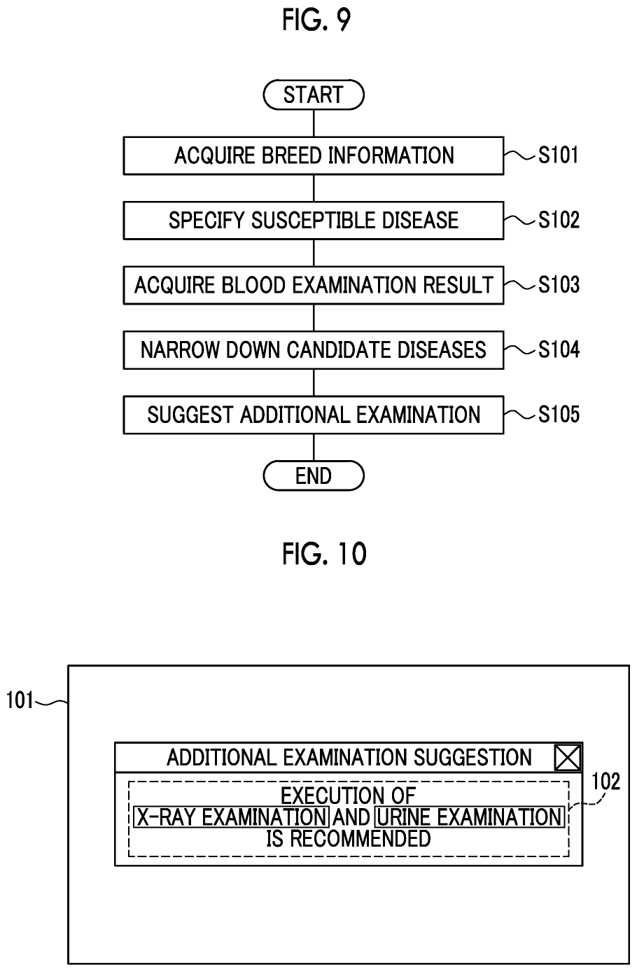

The animal health checkup support system 10 configured as above operates as shown in FIG. 9. In a case where the animal hospital 11 has a visit from an animal as an affected animal, and registers an ID and a breed of the affected animal in the information terminal 17, the animal health checkup support system 10 acquires at least breed information, for example, from the information terminal 17 (Step S101), and specifies susceptible diseases of the breed (Step S102). Thereafter, in a case where, for example, a blood examination as a general-purpose examination is executed, the animal health checkup support system 10 acquires a result of the blood examination (Step S103), and narrows down candidate diseases from a range including the susceptible diseases (Step S104). Then, decision is made whether or not an additional examination is necessary on diagnosis of the candidate diseases, and in a case where the additional examination is necessary, the additional examination for use in the diagnosis of the candidate diseases is suggested (Step S105).

The suggestion of the additional examination is performed, for example, as shown in FIG. 10, by displaying a message 102 for suggesting the additional examination on the display unit 101 of the information terminal 17. The message 102 of FIG. 10 is a message for suggesting execution of an X-ray examination and a urine examination as the additional examination. In a case where the veterinarian views the message 102, the veterinarian can easily recognize an additional examination necessary for diagnosis of doubtful diseases taking an animal species, a breed, symptoms, examination results, and the like into consideration. Furthermore, since the suggestion of the additional examination suggested by the animal health checkup support system 10 takes not only the result of the blood examination but also the breed into consideration, susceptible diseases of the breed are considered without omission, and likelihood is high. For this reason, the animal health checkup support system 10 can perform support pertaining to diagnosis with high likelihood or the like in a case of diagnosing a health state or the like of an animal.

In the above-described first embodiment, although the additional examination suggestion unit 54 displays the message 102 for notifying of an examination to be additionally executed, in a case of an examination capable of obtaining examination results on a plurality of items, such as a blood examination, it is preferable to designate items of which examination results should be additionally obtained. This is to allow the veterinarian to easily determine items on which an additional examination should be executed. Furthermore, in a case of suggesting an additional examination using a message, it is preferable that the additional examination suggestion unit 54 highlights an additional examination to be executed (in FIG. 10, “X-ray examination” and “urine examination”) like the message 102. This is to allow the veterinarian to definitely and easily recognize the additional examination to be executed.

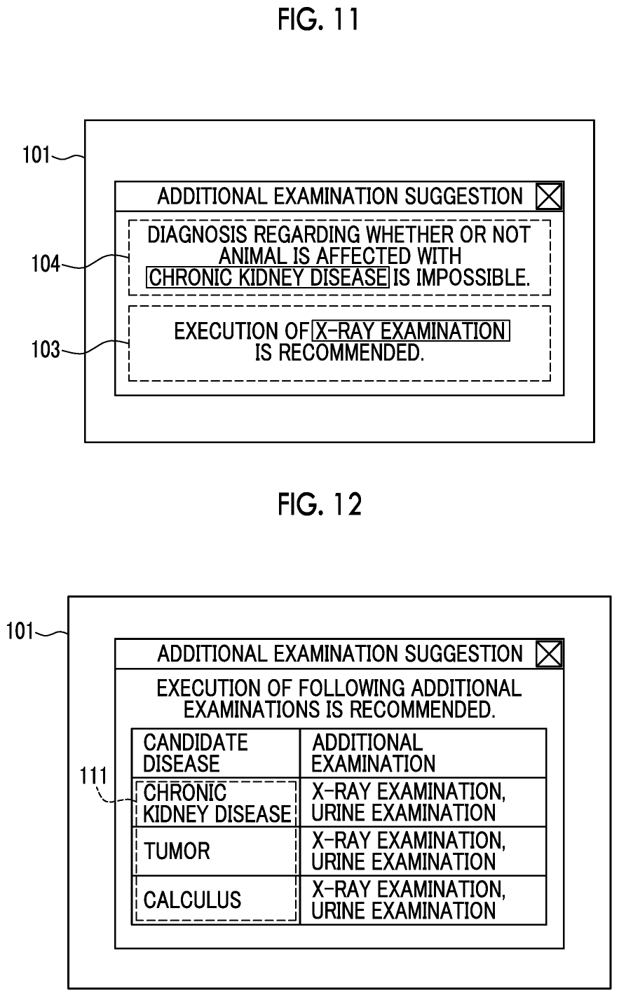

It is preferable that the additional examination suggestion unit 54 notifies of a candidate disease on which diagnosis regarding whether or not an animal is affected with the candidate disease is impossible, and suggests an additional examination on the notified candidate disease. For example, as shown in FIG. 11, the additional examination suggestion unit 54 shows a second message 104 indicating a candidate disease (in FIG. 11, “chronic kidney disease”) on which diagnosis regarding whether or not the animal is affected with the candidate disease is impossible, along with a first message 103 indicating a classification of an examination to be additionally executed. Specifically, in a case where an animal as a target of examination and diagnosis is a cat, there is an abnormal increase in BUN, Cre, SDMA, or the like as a marker of a kidney function in the result of the blood examination, diagnosis regarding whether the cause of the abnormal increase is a tumor, a chronic kidney disease, calculus, or the like is impossible. For this reason, for example, as described above, like the second message 104, one or all candidate diseases on which diagnosis is impossible are presented, and the veterinarian is notified of the effect that diagnosis regarding whether or not the animal is affected with the candidate disease is impossible. Accordingly, the above-described second message 104 is shown, whereby the veterinarian definitely and easily recognizes doubtful diseases. Furthermore, the veterinarian also definitely and easily recognizes a meaning of executing an additional examination (in FIG. 11, “X-ray examination”) notified using the first message 103.

In a case where the candidate disease specification unit 53 specifies a plurality of candidate diseases, the additional examination suggestion unit 54 can suggest an additional examination for each candidate disease. For example, as shown in FIG. 12, a list 111 of candidate diseases is shown, and an additional examination (in a case where there are a plurality of examinations, a list of examinations to be additionally executed) necessary for diagnosis is suggested for each candidate disease. In this way, the additional examination is suggested for each candidate disease on a plurality of candidate diseases specified by the candidate disease specification unit 53, whereby the veterinarian definitely and easily recognizes a doubtful disease and an additional examination necessary for diagnosis of the doubtful disease in association with each other taking the breed and the examination results into consideration.

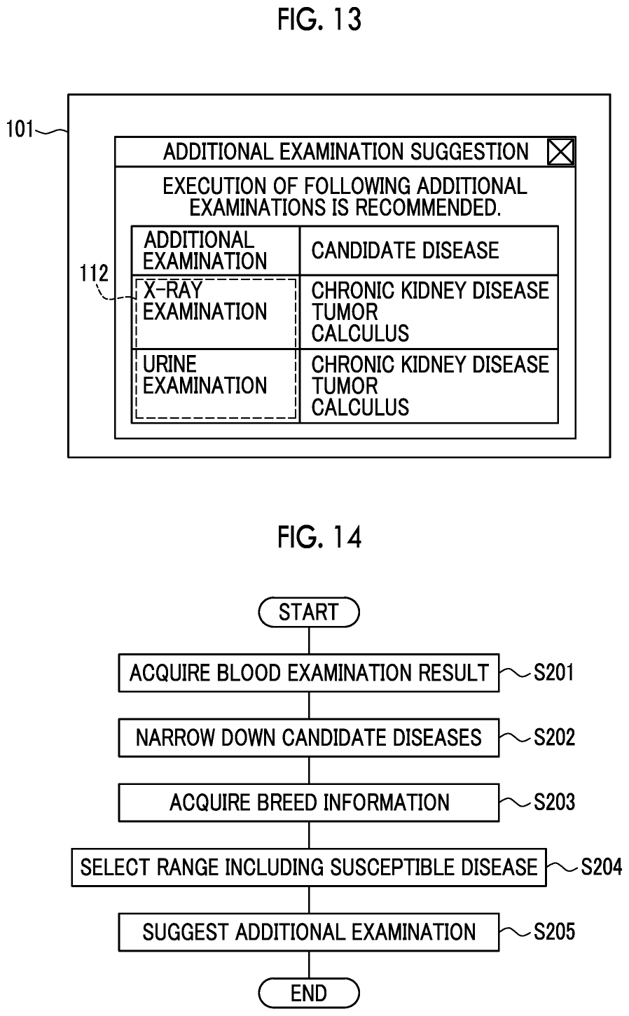

Furthermore, the additional examination suggestion unit 54 can notify of a candidate disease on which diagnosis becomes possible with a suggested additional examination and can suggest the additional examination that makes diagnosis of the notified candidate disease possible. For example, this corresponds to an aspect of FIG. 12. Furthermore, as shown in FIG. 13, a list 112 of examinations (additional examinations) to be additionally executed is shown, and a candidate disease on which diagnosis becomes possible is shown for each classification of the additional examination. In the examples of FIGS. 12 and 13, since the candidate disease on which diagnosis becomes possible with the execution of the suggested additional examination, the additional examination that should be executed to make diagnosis of each candidate disease possible is definite, whereby the veterinarian easily recognize a meaning of the additional examination that is suggested by the animal health checkup support system 10.

In the above-described first embodiment, the candidate disease specification unit 53 specifies the susceptible diseases using the breed information, and then, narrows down the candidate disease from the range including the susceptible disease using the result of the blood examination. In contrast, the candidate diseases are narrowed down using the result of the blood examination, and then, the range including the susceptible diseases is selected from among the narrowed-down candidate diseases, whereby it is possible to specify the candidate disease within the range including the susceptible diseases. That is, as shown in FIG. 14, in a case where the result of the blood examination is registered in, for example, the information terminal 17, the animal health checkup support system 10 acquires the result of the blood examination from the information terminal 17 (Step S201), and narrows down the candidate diseases from all diseases with which the animal can be affected (Step S202). On the other hand, the breed information is acquired (Step S203), and the range including the susceptible diseases is selected from the candidate diseases narrowed down using the result of the blood examination (Step S204). Then, decision is made whether or not an additional examination is necessary on the diagnosis of the candidate diseases, and in a case where the additional examination is necessary, the additional examination for use in the diagnosis of the candidate diseases is suggested (Step S205). There may be many candidate diseases narrowed down using the result of the blood examination; however, as described above, in a case where the candidate diseases are narrowed down using the result of the blood examination, and the range including the susceptible diseases is selected from among the narrowed-down candidate diseases, even though there are many candidate diseases narrowed down using the result of the blood examination, range selection according to the breed is performed with subsequent selection of the range including the susceptible disease. Thus, an additional examination that is particularly necessary can be accurately suggested with priority. That is, in a case where the candidate diseases are narrowed down using the result of the blood examination, and then, the range including the susceptible diseases is selected from among the narrowed-down candidate diseases, the animal health checkup support system 10 can perform support pertaining to diagnosis with high likelihood or the like in a case of diagnosing a health state or the like of an animal.

### Second Embodiment

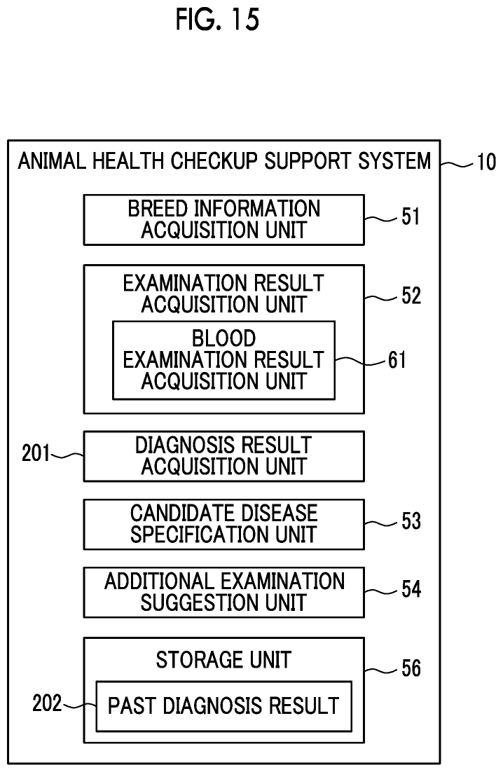

Although the animal health checkup support system 10 of the first embodiment and the modification example described above suggests the additional examination using the breed of the animal as a target of examination and diagnosis and the result of the blood examination executed on the animal, the animal health checkup support system 10 can take a result (hereinafter, referred to as a past diagnosis result) of past diagnosis in a case of suggesting an additional examination. In this case, as shown in FIG. 15, the animal health checkup support system 10 further comprises a diagnosis result acquisition unit 201. The diagnosis result acquisition unit 201 acquires a past diagnosis result pertaining to an animal as a target of examination and diagnosis and past diagnosis results pertaining to other animals (other animals of different animal species or breeds). The past diagnosis result is the content (name, progression degree, and the like of a disease) of diagnosis recorded in, for example, a medical chart or the like, and includes an examination result of a blood examination and the like executed in the past. Furthermore, the storage unit 56 stores a past diagnosis result 202 acquired by the diagnosis result acquisition unit 201 in the database pertaining to the diseases in association with each other. That is, the storage unit 56 stores past diagnosis results as results of diagnosis performed in the past on at least animals of the same or similar breeds to an animal as a target of examination and diagnosis. Then, in a case of specifying candidate diseases, the candidate disease specification unit 53 specifies the candidate diseases with reference to the past diagnosis results pertaining to the animals of the same or similar breeds by referring to the past diagnosis result 202 of the storage unit 56. In this way, the candidate diseases are specified with reference to the past diagnosis results pertaining to the animals of the same or similar breeds, whereby the likelihood of the additional examination to be suggested is improved. In particular, in a case where the breed of the animal as a target of examination and diagnosis is a mongrel with the clarified content of mixing (the breeds of parents or the like), it is possible to increase the likelihood of the additional examination to be suggested.

A range of similarity to a breed can be predetermined at least one of by genetic information pertaining to the breed, empirically, or the like. Furthermore, in a case where there is the past diagnosis result 202 on the animal of the same breed, the candidate disease specification unit 53 specifies the candidate diseases with reference to the past diagnosis result 202 on the animal of the same breed, and in a case where there is no past diagnosis result on the animal of the same breed, the candidate disease specification unit specifies the candidate diseases with reference to the past diagnosis result 202 on the animal of the similar breed. With this, even though the past diagnosis result 202 is accumulated enormously, it is possible to refer to the past diagnosis result 202 in an appropriate range, and to quickly perform specification of the candidate diseases and the suggestion of the additional examination.

As described above, in a case of performing diagnosis support using the past diagnosis result 202, the additional examination suggestion unit 54 can notify of at least one of diagnosis support being performed based on the past diagnosis result 202 or reliability by displaying, for example, a message “A possibility of calculus is XX % from the past tendency. Please additionally execute an X-ray examination and check.”, or the like. Since the ground and the probability of such notification are definite, the veterinarian who receives diagnosis support is likely to reasonably receive the diagnosis support of the animal health checkup support system 10.

### Third Embodiment

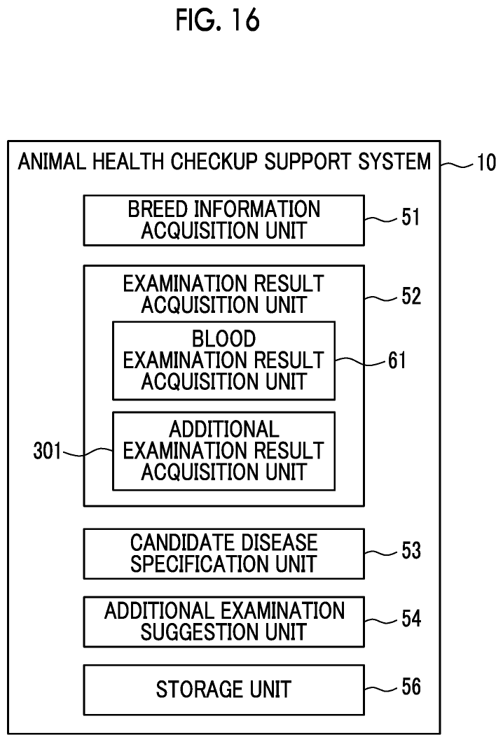

In the first embodiment, the second embodiment, and the modification examples described above, although the animal health checkup support system 10 performs the suggestion of the additional examination, in a case where the additional examination suggested by the animal health checkup support system 10 is executed or in a case where another examination (an additional examination not suggested by the animal health checkup support system 10) is performed, it is possible to perform diagnosis support, such as a suggestion of another additional examination, using an examination result of the additional examination. In this case, as shown in FIG. 16, the examination result acquisition unit 52 further comprises an additional examination result acquisition unit 301. The additional examination result acquisition unit 301 monitors an output of new examination results of various examination devices of the examination device group 16 registration of new examination results in the information terminal 17 after the additional examination suggestion unit 54 performs the suggestion of the additional examination, and in a case where the additional examination suggested by the additional examination suggestion unit 54 is executed or in a case where another new additional examination is executed, acquires a result of the additional examination. Then, the candidate disease specification unit 53 specifies candidate diseases using the result of the additional examination acquired by the additional examination result acquisition unit 301 in addition to the breed information of the result of the blood examination, and the additional examination suggestion unit 54 suggests an additional examination for use in diagnosis regarding whether or not the animal is affected with the candidate diseases specified using the result of the new additional examination. In this way, in a case where the additional examination is executed, a suggestion of an additional examination is newly performed using the result of the additional examination, whereby it is possible to perform a suggestion of an appropriate additional examination sequentially each time an examination is performed.

In the third embodiment, although the suggestion of another additional examination is performed using the new examination result, it is possible to perform diagnosis support by displaying the diagnosis content (whether or not the animal is affected with a specific candidate disease, a progression degree of the specific candidate disease, and the like) of the candidate disease, on which diagnosis becomes possible, on the information terminal 17 instead of or along with the suggestion of another additional examination. In this case, the veterinarian can appropriately receive support for the specific determination content of diagnosis and can smoothly execute a medical examination each time an additional examination is executed.

### Fourth Embodiment

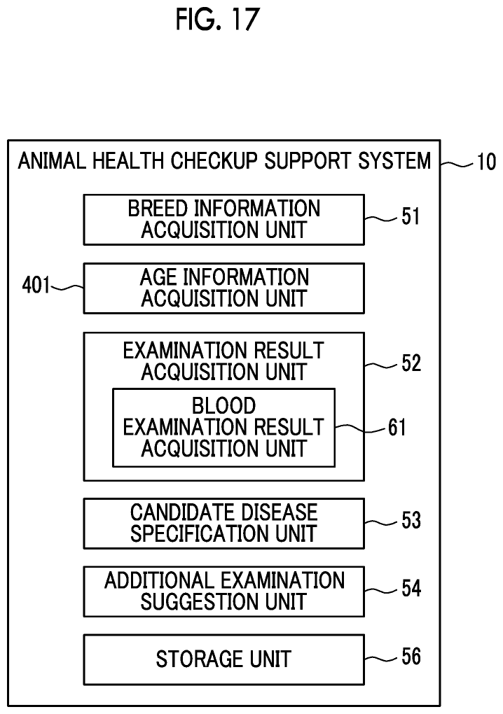

In the first embodiment, the second embodiment, the third embodiment, and the modification examples described above, the animal health checkup support system 10 can take the age of the animal as a target of examination and diagnosis into consideration in a case of performing diagnosis support. In this case, as shown in FIG. 17, the animal health checkup support system 10 comprises an age information acquisition unit 401. The age information acquisition unit 401 acquires information pertaining to the age of the animal as a target of examination and diagnosis. Information (hereinafter, referred to as age information) pertaining to the age is postnatal months, date of birth, or information capable of specifying other than a numerical value representing the age. The age information acquisition unit 401 acquires the age information from, for example, the information terminal 17. Then, the candidate disease specification unit 53 specifies the candidate diseases using the age information in addition to the breed information and the result of the blood examination. In a case where the age information is used in the specification of the candidate disease (consequently, the suggestion of the additional examination), even though an affection rate of the animal as a target of examination and diagnosis with a specific susceptible disease or the like is raised or falls according to the age, it is possible to appropriately perform a suggestion of an additional examination.

The first embodiment, the second embodiment, the third embodiment, the fourth embodiment, and the modification examples can be discretionarily used in combination wholly or partially.

In the first embodiment, the second embodiment, the third embodiment, the fourth embodiment, and the modification examples, although the animal health checkup support system 10 is provided independently of the animal hospital 11, and performs diagnosis support in cooperation with equipment of the animal hospital 11, the animal health checkup support system 10 can be provided in a specific animal hospital 11. For example, the entire animal health checkup support system 10 can be provided in the information terminal 17 of the animal hospital 11. In this case, the animal health checkup support system 10 provided in the specific animal hospital 11 can acquire information (for example, data registered in the database of the storage unit 56, or the like) necessary for diagnosis support in cooperation with equipment of other animal hospitals 11 and can perform diagnosis support in the animal hospital 11 where the animal health checkup support system 10 is provided, other animal hospitals 11 where another animal health checkup support system 10 is provided, and other animal hospitals 11 where no animal health checkup support system 10 is provided, in addition to performing diagnosis support in cooperation with the equipment of the animal hospital 11. The animal health checkup support system 10 can be a single device (one computer or the like that realizes the animal health checkup support system 10). Furthermore, the animal health checkup support system 10 can be provided in a form in which the animal health checkup support system 10 is divided into a plurality of devices that execute a part of functions or processes, and a plurality of devices function as the animal health checkup support system 10 as the whole.

Furthermore, in the first embodiment, the second embodiment, the third embodiment, the fourth embodiment, and the modification examples described above, although the animal health checkup support system 10 performs diagnosis support called the suggestion of the additional examination, using the result of the blood examination in addition to the breed information, the animal health checkup support system 10 can perform initial diagnosis support using results of other examinations, such as a result (at least one of an X-ray captured image, interpretation findings, or the like) of an X-ray examination, instead of the result of the blood examination. This is useful in a case where diagnosis support is performed on a disease (for example, see FIG. 6) that is hardly diagnosed using the result of the blood examination, and enables to perform appropriate diagnosis support. From the beginning, it is possible to perform initial diagnosis support using results of two or more kinds of examinations, such as a result of a blood examination and a result of an X-ray examination. The animal health checkup support system 10 can perform diagnosis support by providing information regarding a diagnosis result of a candidate disease diagnosable at this point of time (whether or not the animal is affected with the candidate disease, or the like) instead of or along with the suggestion of the additional examination.

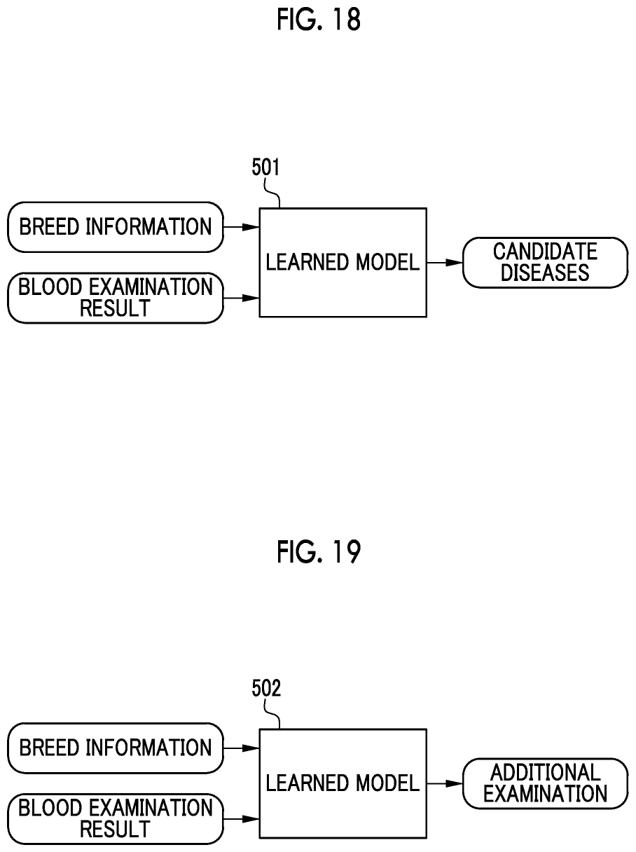

In the first embodiment, the second embodiment, the third embodiment, the fourth embodiment, and the modification examples described above, the candidate disease specification unit 53 and the additional examination suggestion unit 54 can be mounted by a learned model (so-called artificial intelligence (AI) program). For example, as shown in FIG. 18, the candidate disease specification unit 53 can be realized a learned model 501 that receives an input of breed information and a result of a blood examination and outputs candidate diseases. Furthermore, as shown in FIG. 19, the candidate disease specification unit 53 and the additional examination suggestion unit 54 can be realized by a learned model 502 that receives an input of breed information and a result of a blood examination and outputs a necessary additional examination.

The first embodiment, the second embodiment, the third embodiment, the fourth embodiment, and the modification examples described above includes an animal health checkup support method comprising a breed information acquisition step in which a breed information acquisition unit acquires information pertaining to a breed of an animal as a target of examination and diagnosis, a blood examination result acquisition step in which a blood examination result acquisition unit acquires a result of a blood examination of the animal, a candidate disease specification step in which a candidate disease specification unit specifies candidate diseases as candidates of diseases, with which the animal is affected, using information pertaining to the breed and the result of the blood examination, and an additional examination suggestion step in which an additional examination suggestion unit suggests an additional examination for use in diagnosis regarding whether or not the animal is affected with the candidate diseases specified by the candidate disease specification unit. Furthermore, the embodiments include an animal health checkup support device in which a processor, such as a CPU, is configured to acquire information pertaining to a breed of an animal as a target of examination and diagnosis, acquire a result of a blood examination of the animal, specify candidate diseases as candidates of diseases, with which the animal is affected, using information pertaining to the breed and the result of the blood examination, and suggest an additional examination for use in diagnosis regarding whether or not the animal is affected with the candidate diseases specified by the candidate disease specification unit, an animal health checkup support program, and an animal health checkup support method.

In the above-described embodiments and the modification examples, the hardware structures of the processing units that execute various kinds of processing, such as the breed information acquisition unit 51, the examination result acquisition unit 52, the blood examination result acquisition unit 61, the candidate disease specification unit 53, the additional examination suggestion unit 54, the diagnosis result acquisition unit 201, the additional examination result acquisition unit 301, and the age information acquisition unit 401, are various processors described below. Various processors include a central processing unit (CPU) that is a general-purpose processor executing software (program) to function as various processing units, a programmable logic device (PLD) that is a processor capable of changing a circuit configuration after manufacture, such as a graphical processing unit (GPU) or a field programmable gate array (FPGA), a dedicated electric circuit that is a processor having a circuit configuration dedicatedly designed for executing various kinds of processing, and the like.

One processing unit may be configured of one of various processors described above or may be configured of a combination of two or more processors (for example, a plurality of FPGAs, a combination of a CPU and an FPGA, or a combination of a CPU and a GPU) of the same type or different types. A plurality of processing units may be configured of one processor. As an example where a plurality of processing units are configured of one processor, first, as represented by a computer, such as a client or a server, there is a form in which one processor is configured of a combination of one or more CPUs and software, and the processor functions as a plurality of processing units. Secondly, as represented by system on chip (SoC) or the like, there is a form in which a processor that realizes all functions of a system including a plurality of processing units into one integrated circuit (IC) chip is used. In this way, various processing units are configured using one or more processors among various processors described above as a hardware structure.

In addition, the hardware structure of various processors is, more specifically, an electric circuit (circuitry), in which circuit elements, such as semiconductor elements, are combined. Furthermore, the hardware structure of the storage unit is a storage device, such as a hard disc drive (HDD) or a solid state drive (SSD).

### EXPLANATION OF REFERENCES

10: animal health checkup support system
11: animal hospital
16: examination device group
17: information terminal
21: blood examination device
22: X-ray examination device
23: ultrasound examination device
24: MRI examination device
25: CT examination device
31: urine examination device
32: fecal examination device
33: eye examination device
34: oral examination device
35: skin examination device
51: breed information acquisition unit
52: examination result acquisition unit
53: candidate disease specification unit
54: additional examination suggestion unit
56: storage unit
61: blood examination result acquisition unit
101: display unit
102: message
103: first message
104: second message
111: list
112: list
201: diagnosis result acquisition unit
202: past diagnosis result
301: additional examination result acquisition unit
401: age information acquisition unit
501: learned model
502: learned model
A1 to A8: disease
C1 to C3: breed
S101 to S205: operation step

What is claimed is:

1. An animal health checkup support system comprising:
a processor configured to function as:
a breed information acquisition unit that acquires information pertaining to a breed of an animal as a target for examination and diagnosis;
a blood examination result acquisition unit that acquires a result of a blood examination of the animal;
a candidate disease specification unit that specifies candidate diseases as candidates of diseases, with which the animal is affected, using information pertaining to the breed and the result of the blood examination; and
an additional examination suggestion unit that suggests an additional examination for use in diagnosis regarding whether or not the animal is affected with the candidate diseases specified by the candidate disease specification unit.

2. The animal health checkup support system according to claim 1,
wherein the candidate disease specification unit specifies the candidate diseases within a range including at least a susceptible disease by specifying the susceptible disease of the animal using information pertaining to the breed and narrowing down the candidate diseases using the result of the blood examination.

3. The animal health checkup support system according to claim 2,
wherein the candidate disease specification unit specifies the candidate diseases within the range including the susceptible disease by selecting the candidate diseases using the result of the blood examination from within the range including the susceptible disease.

4. The animal health checkup support system according to claim 2,
wherein the candidate disease specification unit specifies the candidate diseases within the range including the susceptible disease by selecting the range including the susceptible disease from among the candidate diseases.

5. The animal health checkup support system according to claim 1,
wherein, in a case where the candidate disease specification unit specifies a plurality of the candidate diseases, the additional examination suggestion unit suggests an additional examination for each candidate disease.

6. The animal health checkup support system according to claim 2,
wherein, in a case where the candidate disease specification unit specifies a plurality of the candidate diseases, the additional examination suggestion unit suggests an additional examination for each candidate disease.

7. The animal health checkup support system according to claim 3,
wherein, in a case where the candidate disease specification unit specifies a plurality of the candidate diseases, the additional examination suggestion unit suggests an additional examination for each candidate disease.

8. The animal health checkup support system according to claim 4,
wherein, in a case where the candidate disease specification unit specifies a plurality of the candidate diseases, the additional examination suggestion unit suggests an additional examination for each candidate disease.

9. The animal health checkup support system according to claim 1,
wherein the additional examination suggestion unit notifies of the candidate disease on which diagnosis regarding whether or not the animal is affected with the candidate disease is impossible and suggests an additional examination on the notified candidate disease.

10. The animal health checkup support system according to claim 2,
wherein the additional examination suggestion unit notifies of the candidate disease on which diagnosis regarding whether or not the animal is affected with the candidate disease is impossible and suggests an additional examination on the notified candidate disease.

11. The animal health checkup support system according to claim 3,
wherein the additional examination suggestion unit notifies of the candidate disease on which diagnosis regarding whether or not the animal is affected with the candidate disease is impossible and suggests an additional examination on the notified candidate disease.

12. The animal health checkup support system according to claim 4,
wherein the additional examination suggestion unit notifies of the candidate disease on which diagnosis regarding whether or not the animal is affected with the candidate disease is impossible and suggests an additional examination on the notified candidate disease.

13. The animal health checkup support system according to claim 1,
wherein the additional examination suggestion unit notifies of the candidate disease on which diagnosis becomes possible with an additional examination to be suggested and suggests the additional examination that makes diagnosis of the notified candidate disease possible.

14. The animal health checkup support system according to claim 2,
wherein the additional examination suggestion unit notifies of the candidate disease on which diagnosis becomes possible with an additional examination to be suggested and suggests the additional examination that makes diagnosis of the notified candidate disease possible.

15. The animal health checkup support system according to claim 3,
wherein the additional examination suggestion unit notifies of the candidate disease on which diagnosis becomes possible with an additional examination to be suggested and suggests the additional examination that makes diagnosis of the notified candidate disease possible.

16. The animal health checkup support system according to claim 4,
wherein the additional examination suggestion unit notifies of the candidate disease on which diagnosis becomes possible with an additional examination to be suggested and suggests the additional examination that makes diagnosis of the notified candidate disease possible.

17. The animal health checkup support system according to claim 1, further comprising:
a storage that stores a past diagnosis result as a result of past diagnosis on the animal of the same or similar breed,
wherein the candidate disease specification unit specifies the candidate diseases with reference to the past diagnosis result.

18. The animal health checkup support system according to claim 17,
wherein, in a case where there is the past diagnosis result on the animal of the same breed, the candidate disease specification unit specifies the candidate diseases with reference to the past diagnosis result on the animal of the same breed, and
in a case where there is no past diagnosis result on the animal of the same breed, the candidate disease specification unit specifies the candidate diseases with reference to the past diagnosis result on the animal of the similar breed.

19. The animal health checkup support system according to claim 1,
wherein the processor is further configured to function as an additional examination result acquisition unit that, in a case where an additional examination suggested by the additional examination suggestion unit is executed, acquires a result of the additional examination,
the candidate disease specification unit specifies the candidate disease using the result of the additional examination in addition to information pertaining to the breed and the result of the blood examination, and
the additional examination suggestion unit suggests an additional examination for use in diagnosis regarding whether or not the animal is affected with the candidate diseases specified using the result of the additional examination.
